(19) United States
(12) Patent Application Publication
(10) Pub. No.: US 2024/0191296 A1
(43) Pub. Date: Jun. 13, 2024

(54) SPECIFIC DNA FRAGMENTS FOR SEX IDENTIFICATION OF MASTACEMBELUS ARMATUS, GENETIC sex MARKER PRIMERS AND APPLICATIONS THEREOF

(71) Applicant: Guangzhou University, Guangzhou City (CN)

(72) Inventors: Hu Shu, Guangzhou City (CN); Chong Han, Guangzhou City (CN); Weijian Qin, Guangzhou City (CN); Mingxiang Cui, Guangzhou City (CN); Jinlin Yang, Guangzhou City (CN); Baoyue Lu, Guangzhou City (CN); Zhide Yu, Guangzhou City (CN); Jiaxing Cui, Guangzhou City (CN)

(21) Appl. No.: 18/538,550

(22) Filed: Dec. 13, 2023

(30) Foreign Application Priority Data
Dec. 13, 2022 (CN) 2022115949004

(51) Int. Cl. C12Q 1/6879

(52) U.S. Cl. CPC C12Q 1/6879

(57) ABSTRACT

A specific DNA fragment, genetic sex marker primer, and application for identifying the sex of Mastacembelus armatus. The specific DNA fragment includes upstream primer of Contig-1 with a nucleotide sequence shown as SEQ ID NO.1, downstream primer of Contig-1 with a nucleotide sequence shown as SEQ ID NO.2, upstream primer of Contig-2 with a nucleotide sequence shown as SEQ ID NO.3, and downstream primer of Contig-2 with a nucleotide sequence shown as SEQ ID NO.4. The genetic sex marker primer for identifying the sex of Mastacembelus armatus is primer pair 1, including upstream primer of Contig-1 and downstream primer of Contig-1. This specific DNA fragment and genetic sex marker primer can be applied to the all-male breeding of Mastacembelus armatus. This application has a wide range of applications, a short detection time, and accurate results.

### REFERENCE TO AN ELECTRONIC SEQUENCE LISTING

The content of the electronic sequence listing (Substitute Sequence Listing.xml; Size: 10,138 bytes; and Date of Creation: Jan. 30, 2024) is herein incorporated by reference in its entirety.

### TECHNICAL FIELD

This application belongs to the genetic and fish sex identification technology, specifically involving a specific DNA fragment for the sex identification of the Mastacembelus armatus, genetic sex marker primers, and their applications.

### BACKGROUND

In recent years, with the development of sequencing technology, the sex markers for many animals have been widely used, such as Nile tilapia (Oreochromis niloticus), half-smooth tongue sole (Cynoglossus semilaevis), and rainbow trout (Oncorhynchus mykiss). However, the development of these molecular markers is mainly obtained through techniques such as Random Amplified Polymorphic DNA (RAPD), Simple Sequence Repeat (SSR), and Single Nucleotide Polymorphic (SNP), which are expensive and labor-intensive. The resequencing technology provides an efficient means for the development of genetic sex markers. Specific molecular markers for sex identification in Mastacembelus armatus have been successfully developed using resequencing technology.

The Mastacembelus armatus belongs to the genus Mastacembelus of the family Mastacembelidae of the order Synbranchiformes. It is a freshwater fish commonly known as the tire-track spiny eel or zig-zag eel, mainly found in several Southeast Asian countries as well as in Guangdong, Guangxi, Fujian, and Hainan provinces of China. It is highly nutritious and has excellent meat quality, making it popular among consumers. The growth process of the Mastacembelus armatus exhibits sexual dimorphism, with male fish weighing more than 20% heavier than female fish of the same age. This makes the all-male mono-sex breeding of the Mastacembelus armatus more economically valuable.

The specific DNA segments for sex identification of the Mastacembelus armatus have been disclosed in Chinese application application NO. 202010057366.8. Based on the 2b-RAD-seq technique and using the genome walking technique, the application has developed male-specific sex markers for the Mastacembelus armatus. The specific DNA segments are shown as SEQ ID NO.1 or SEQ ID NO.2. With these specific sequences, sex identification of the Mastacembelus armatus can be carried out using conventional PCR amplification and agarose gel electrophoresis. The primers used for sex identification of the Mastacembelus armatus are CTACACAGGCAATACTTGGCAAATGAATAC and ATCAGTCATCTGTGCCTGGGATATATG. However, the technique proposed in the document has the following limitations: firstly, the sequencing method used can only be 2b-RAD-seq, suitable for the F1 offspring population of breeding families, with the marker band criteria being: female: no band, male: 431 bp/746 bp. It can only accurately determine the sex of males, but not of females or both sexes and cannot accurately determine a wider range of wild and breeding populations, making it difficult to apply to the widespread sex identification and breeding of the Mastacembelus armatus.

In addition, according to the known genome sequence of the Mastacembelus armatus, it was found that it has a more obvious sex chromosome. Our team analyzed the sequencing of the male and female genomes of the Mastacembelus armatus compared it with the genome sequence, and found that the male genome has many specific DNA segments. Therefore, it is speculated that the sex determination of the Mastacembelus armatus is male heterozygous, and it is difficult to accurately determine its sex using a single specific DNA segment. The first step in the all-male breeding of the Mastacembelus armatus requires obtaining pseudo-female fish, and the main way to obtain pseudo-female fish is through inducing sex reversal in male fish. Whether it is through observing the external morphology of the Mastacembelus armatus or using traditional cross-breeding methods to identify the parents, it requires a lot of time and manpower. The existing markers for the Mastacembelus armatus are only a kind of universal marker for the lineage, and only show bands in male individuals, which is quite limiting. Therefore, the development of an intuitive, accurate, and widely applicable molecular marker and method for identifying genetic males, and its application in the all-male breeding of the Mastacembelus armatus, with large-scale promotion and popularization, has significant practical significance.

### SUMMARY

The purpose of this application is to address the shortcomings of existing technology by providing a specific DNA fragment for sex identification of Mastacembelus armatus, genetic sex marker primers, and their application. This new approach aims to offer a highly stable genetic sex marker primer that can be used for multiple populations of Mastacembelus armatus, allowing for the identification of male and female Mastacembelus armatus at the molecular level. Additionally, the method for sex identification of Mastacembelus armatus is improved to meet the requirements of wide applicability, short time consumption, and accurate detection results in the production process.

To achieve the first objective mentioned above, the present application adopts the following technical solution.

A specific DNA fragment for sex identification of Mastacembelus armatus, wherein one of the following specific DNA fragments: upstream primer of Contig-1, with a nucleotide sequence as shown in SEQ ID NO.1; downstream primer of Contig-1, with a nucleotide sequence as shown in SEQ ID NO.2; upstream primer of Contig-2, with a nucleotide sequence as shown in SEQ ID NO.3; downstream primer of Contig-2, with a nucleotide sequence as shown in SEQ ID NO.4.

| | | product | | | |
|---|---|---|---|---|---|
| primer | | length | | primer | Tm |
| name | primer ID | (bp) | primer sequence (5′-3′) | length | (° C.) |
| Contig-1 | SEQ ID NO. 1 | 354/473 | CCATTCAGCATTACACCATACA | 22 | 53 |
| | (DCQ-1F) | | | | |
| | SEQ ID NO. 2 | | TTCCCCTACCAAATCACCA | 19 | |
| | (DCQ-1R) | | | | |
| Contig-2 | SEQ ID NO. 3 | 649/491 | TGCTACTGTCCAAAGTAAACAGG | 23 | 54 |
| | (DCQ-2F) | | | | |
| | SEQ ID NO. 4 | | CGGGCAGAATCACTCAAA | 18 | |
| | (DCQ-2R) | | | | |

A genetic sex marker primer for sex identification of Mastacembelus armatus, which consists of primer pair 1, including the upstream primer of Contig-1 and the downstream primer of Contig-1.

The nucleotide sequences of primer pair 1 from 5′-3′ are as follows.

Contig-1 upstream primer:
5′-CCATTCAGCATTACACCATACA-3′;
Contig-1 downstream primer:
5′-TTCCCCTACCAAATCACCA-3′.

The genetic sex marker primer also includes primer pair 2, comprising the upstream primer of Contig-2 and the downstream primer of Contig-2.

The nucleotide sequences of primer pair 2 from 5′-3′ are as follows.

Contig-2 upstream primer:
5′-TGCTACTGTCCAAAGTAAACAGG-3′;
Contig-2 downstream primer:
5′-CGGGCAGAATCACTCAAA-3′.

A method for sex identification of Mastacembelus armatus using the aforementioned genetic sex marker primers comprises the following steps.

S1: Designing primer pair 1 and primer pair 2;

S2: Preparing Mastacembelus armatus DNA samples;

S3: Using the DNA sample obtained in S2 as a template, performing PCR amplification with the designed primer pair 1 and primer pair 2 from S1, and detecting the results using gel electrophoresis. If the electrophoresis result shows a single DNA-specific band, the tested Mastacembelus armatus is female; if it shows two DNA-specific bands, the tested Mastacembelus armatus is male.

In S2, the preparation of DNA samples is carried out using a DNA extraction kit for Mastacembelus armatus DNA extraction.

In S3, when performing PCR amplification using primer pair 1 or primer pair 2, the 25 μL PCR reaction system includes 100 ng Mastacembelus armatus DNA sample, 1 μL each of upstream and downstream primers of Contig-1 or Contig-2, 12.5 μL 2×TaqMasterMix, and ddH₂O up to a total volume of 25 μL.

In S3, the PCR reaction program for amplification using primer pairs is as follows: initial denaturation at 95° C. for 3 min; followed by 20 cycles of denaturation at 95° C. for 15 s, annealing at 58° C. for 15 s, and extension at 72° C. for 1 min; then 15 cycles of denaturation at 95° C. for 15 s, annealing at 50° ° C. for 15 s, and extension at 72° C. for 1 min; and a final extension at 72° C. for 5 min.

S3, in the process of electrophoresis detection, using a 1.2% agarose gel to detect the number and length of amplified fragments.

In the electrophoresis result of S3, when a specific band is amplified, the Mastacembelus armatus is female if there is one band, and male if there are two bands. The specific band length of the X chromosome product of primer pair 1 is 354 bp, and the specific band length of the Y chromosome product is 473 bp. The specific band length of the X chromosome product of primer pair 2 is 649 bp, and the specific band length of the Y chromosome product is 491 bp.

An application of the specific DNA fragment for sex identification of Mastacembelus armatus in an all-male breeding program for Mastacembelus armatus as described above.

An application of the aforementioned genetic sex marker primer for all-male breeding of Mastacembelus armatus.

This application has at least the following beneficial effects.

(1) The specific DNA fragment for sex identification of Mastacembelus armatus provided by the present application is based on WGS seq genome sequencing technology and data of female and male Mastacembelus armatus, and is designed based on comprehensive analysis methods such as genome assembly and alignment. It consists of four DNA segments that can complement each other. It has good stability and wide applicability. By combining them in pairs, two sets of molecular marker primers can be formed, which can be widely used to identify the genetic sex of Mastacembelus armatus in various wild and cultured populations in the Pearl River Basin. These primer pairs can rapidly and accurately identify the sex of female and male Mastacembelus armatus simultaneously, and can be used to quickly and accurately select genetic sex markers for Mastacembelus armatus in a wide range and multiple populations in the industrial production process. This can fulfill the requirements of large-scale breeding and production of Mastacembelus armatus.

(2) The method provided by the present application for identifying the genetic sex of Mastacembelus armatus using the specific DNA fragment and its molecular marker primer pairs is based on WGS seq genome sequencing data of female and male Mastacembelus armatus. By using two pairs of specific primers, the sex of Mastacembelus armatus can be identified through PCR reaction of the DNA samples. Compared to traditional sex identification methods, this method is more versatile, less time-consuming, and more efficient and accurate.

(3) The application of the specific DNA fragment and genetic sex marker primers provided by the present application can identify the sex of female and male Mastacembelus armatus at the molecular level. By comprehensively using analysis methods such as genome assembly and alignment, it can be applied to the process of large-scale all-male breeding and production of Mastacembelus armatus in multiple populations, meeting production needs.

### BRIEF DESCRIPTION OF THE DRAWINGS

FIG. 1 shows a schematic diagram of the method for obtaining candidate chromosomal segments of Mastacembelus armatus in the embodiment of the present application.

FIG. 2 shows a schematic diagram of the length distribution of unique candidate segments of Mastacembelus armatus in the embodiment of the present application (the horizontal axis represents the length of candidate segments in bp, and the vertical axis represents the number of candidate segments).

FIG. 3 shows the electrophoresis image of the PCR products of the 2 pairs of primers detecting specific amplification fragments in male and female samples in the embodiment of the present application.

FIG. 4 shows the electrophoresis image of the PCR products detected by primer Contig-1F/R in the wild populations (8 males and 8 females) of the Dongjiang, Beijiang, Xijiang, and Tanjiang in the Pearl River Basin in the embodiment of the present application.

### DETAILED DESCRIPTION OF THE EMBODIMENTS

The following detailed description of the technical solution of the present application is combined with the accompanying drawings 1-4 and specific embodiments, so that those skilled in the art can fully understand the technical solution of the present application. All reagents or materials used in the embodiments of the present application, unless otherwise specified, are from commercial sources.

### Embodiment 1

The specific DNA fragments for sex identification of Mastacembelus armatus provided in this embodiment are as follows: upstream primer of Contig-1, with a nucleotide sequence as shown in SEQ ID NO.1; downstream primer of Contig-1, with a nucleotide sequence as shown in SEQ ID NO.2; upstream primer of Contig-2, with a nucleotide sequence as shown in SEQ ID NO.3; downstream primer of Contig-2, with a nucleotide sequence as shown in SEQ ID NO.4. Refer to the gene sequence list of SEQ ID NO.1, SEQ ID NO.2, SEQ ID NO.3, SEQ ID NO.4 for details.

This embodiment provides genetic sex marker primers including the specific DNA fragments for sex identification of Mastacembelus armatus, which are primer pair 1 and primer pair 2, and both sets of primer pairs can be used separately.

Wherein, primer pair 1 includes the upstream primer of Contig-1 and the downstream primer of Contig-1.

The nucleotide sequences of primer pair 1 from 5′-3′ are as follows.

Upstream primer of Contig-1:
5′-CCATTCAGCATTACACCATACA-3′.
Downstream primer of Contig-1:
5′-TTCCCCTACCAAATCACCA-3′.

The specific nucleotide sequence of the upstream primer of Contig-1 is as shown in SEQ ID NO.1, and the specific nucleotide sequence of the downstream primer of Contig-1 is as shown in SEQ ID NO.2.

Primer pair 2 includes the upstream primer of Contig-2 and the downstream primer of Contig-2.

The nucleotide sequences of primer pair 2 from 5′-3′ are as follows.

Upstream primer of Contig-2:
5′-TGCTACTGTCCAAAGTAAACAGG-3′.
Downstream primer of Contig-2:
5′-CGGGCAGAATCACTCAAA-3′.

The specific nucleotide sequence of the upstream primer of Contig-2 is as shown in SEQ ID NO.3, and the specific nucleotide sequence of the downstream primer of Contig-2 is as shown in SEQ ID NO.4.

The specific DNA fragments for sex identification of Mastacembelus armatus and genetic sex molecular marker primers in the above embodiments of the present application are obtained through the following steps.

### (I) Construction and Re-Sequencing of Sequencing Library

1. Preparing Mastacembelus armatus DNA Samples:

during the breeding period of wild Mastacembelus armatus, 8 males and 8 females were obtained from the Tanjiang, and tail fin samples were randomly taken from 3 females and 3 males, numbered F1-F3 and M1-M3, respectively. The DNA samples were prepared using a conventional column centrifugation method (universal column type genomic extraction kit, from Shanghai MyBio Technology Co., Ltd.), following the instructions, and the quality and concentration were tested using 1.2% agarose gel electrophoresis and a microplate reader (BioTek Instruments, Inc.), meeting the sequencing requirements.

### 2. Construction of Sequencing Library and High-Throughput Sequencing:

DNA samples of F1-F3 and M1-M3 were used to construct 350 bp-500 bp fragment sequencing libraries, and paired-end (PE150) sequencing was performed using the Illumina Hiseq X Ten platform. The library construction steps specifically refer to the Novogene NGS DNA Library Prep Kit instructions, and the sequencing was completed by Novogene Corporation. Clean data for female and male genome sequencing were respectively obtained: 42 Gb and 64 Gb.

### (II) Enrichment of Sex Chromosome-Specific DNA Fragments

### 1. Acquisition of Candidate DNA Fragments

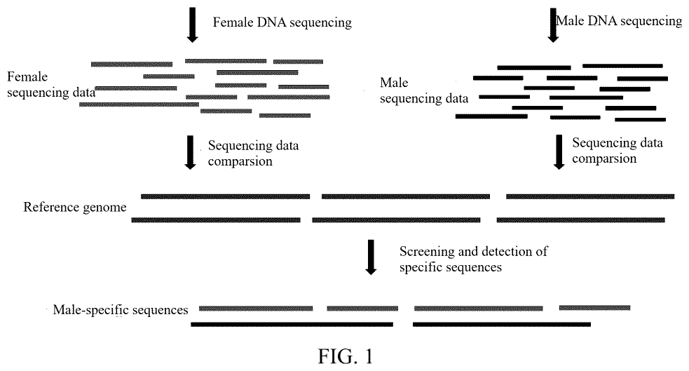

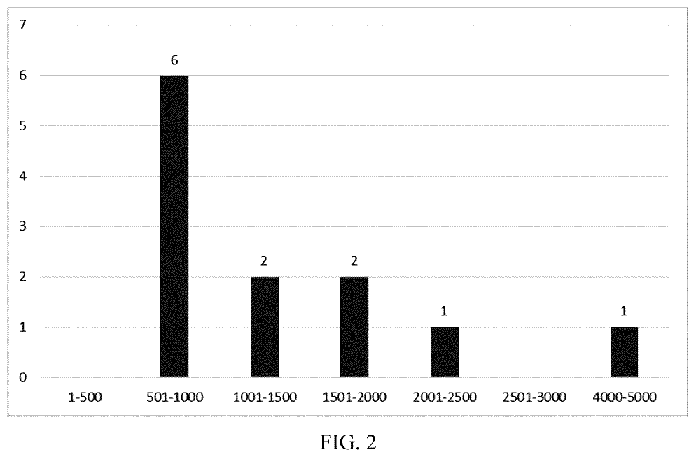

Female fish F1-F3, sequencing reads, and male fish M1-M3 sequencing reads were aligned to the NCBI male loach chromosome genome using the bwa v0.7.17-r1188 (http://bio-bwa.sourceforge.net/) software's aln method. It was found that the male fish sequencing reads could all be aligned, but the female fish sequencing reads could not be aligned in certain regions (see FIG. 1). A total of 6 male-specific DNA fragments were selected, with the lengths of these male fish DNA fragments mainly distributed between 500-1000 bp, accounting for 50.0% of the total; Contigs with a length of ≥300 bp accounted for 100% of the total. “These 6 Contigs contain Y chromosome-specific DNA fragments (see FIG. 2).

### (III) Selection of Male-Specific Molecular Markers

### 1. Primer Design and Synthesis:

Based on the aforementioned 6 male fish DNA sequences, PCR primers were designed using Primer5 software (software usage refers to Primer PREMIER Version5.0 for Windows and Power Macintosh), and the primers were numbered according to DCQ-xxF/R (xx represents the Contig number), such as the primer number for Contig-1 being DCQ-1F/R (F represents the upstream primer, R represents the downstream primer, the same below). The primers were synthesized by Sangon Biotech (Shanghai) Co., Ltd.

### 2. Selection and Confirmation of Male-Specific Molecular Markers:

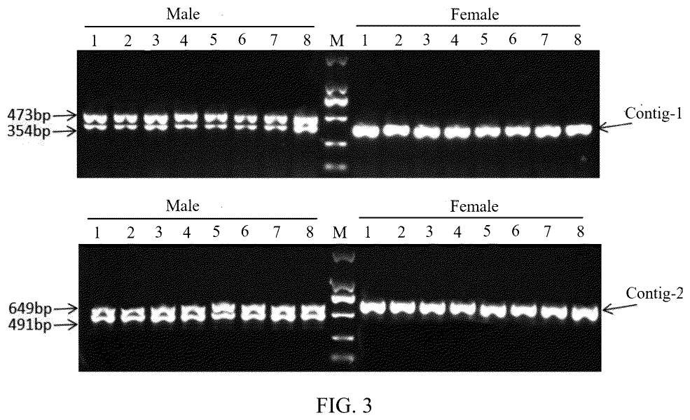

The aforementioned 6 pairs of primers were used to conduct PCR tests on 8 male and 8 female individuals. The overall PCR amplification reaction system was 25 μL, including 12.5 μL 2×Taq MasterMix (Nanjing Novozymes Biotechnology Co., Ltd.), 1 μL of each upstream and downstream primer, 100 ng of template DNA, and ddH₂O added to a total volume of 25 μL. The PCR reaction program was: pre-denaturation at 95° C. for 3 min; followed by denaturation at 95° C. for 15 s, annealing at 58° C. for 15 s, extension at 72° C. for Imin, for a total of 20 cycles; followed by denaturation at 95° C. for 15 s, annealing at 50° C. for 15 s, extension at 72° C. for 1 min, for a total of 15 cycles; and a final extension at 72° C. for 5 min. The agarose gel electrophoresis results showed that only two pairs of primers were able to obtain a single amplification band in female individuals and two amplification bands in male individuals (see FIG. 3), while the rest exhibited non-specific bands and indeterminate smearing bands; the primers were DCQ-1F/R and DCQ-2F/R, corresponding to Contig-1 and Contig-2, respectively.

Through the PCR amplification of these 6 pairs of primers in 8 male and 8 female individuals of Mastacembelus armatus, it was confirmed that 2 Contigs, DCQ-1 and DCQ-2, are sex chromosome-specific DNA fragments and can be used as genetic sex markers for Mastacembelus armatus.

### Embodiment 2

This embodiment provides a method for sex identification of Mastacembelus armatus using the genetic sex markers designed in Embodiment 1, including the following steps.
S1: Designing genetic sex molecular markers for Mastacembelus armatus (primer pairs 1 and 2), referring to the specific operations in Embodiment 1.
S2: Preparation of Mastacembelus armatus DNA samples:
obtaining visually mature wild Mastacembelus armatus species from the Pearl River Basin, including the Dongjiang, Beijiang, Xijiang, and Tanjiang, and dissecting 8 female and 8 male Mastacembelus armatus; cutting off the tail fins and using a general-purpose DNA extraction kit (Guangzhou MyBio Science & Technology Co., Ltd.) with routine column centrifugation according to the instructions to prepare DNA samples.
S3: PCR amplification verification:
using the DNA samples obtained in S2 as templates and performing PCR amplification using primer pairs 1 and 2 designed in S1; after detection by gel electrophoresis, if a single DNA-specific band is present, the Mastacembelus armatus being tested is female; if two DNA-specific bands are present, the Mastacembelus armatus being tested is male.

In this embodiment, the primer pairs DCQ-1F/R and DCQ-2F/R were used to perform PCR validation on the aforementioned 8 female and 8 male Mastacembelus armatus DNA samples. The overall PCR amplification reaction system was 25 μL, including 12.5 μL 2×Taq MasterMix (Nanjing Novozymes Biotechnology Co., Ltd.), 1 μL of each upstream and downstream primer, 100 ng of template DNA, and ddH₂O added to a total volume of 25 μL.

The PCR reaction program was: pre-denaturation at 95° C. for 3 min; followed by denaturation at 95° C. for 15 s, annealing at 58° C. for 15 s, extension at 72° C. for 1 min, for a total of 20 cycles; followed by denaturation at 95° C. for 15 s, annealing at 50° C. for 15 s, extension at 72° C. for 1 min, for a total of 15 cycles; and a final extension at 72° C. for 5 min.

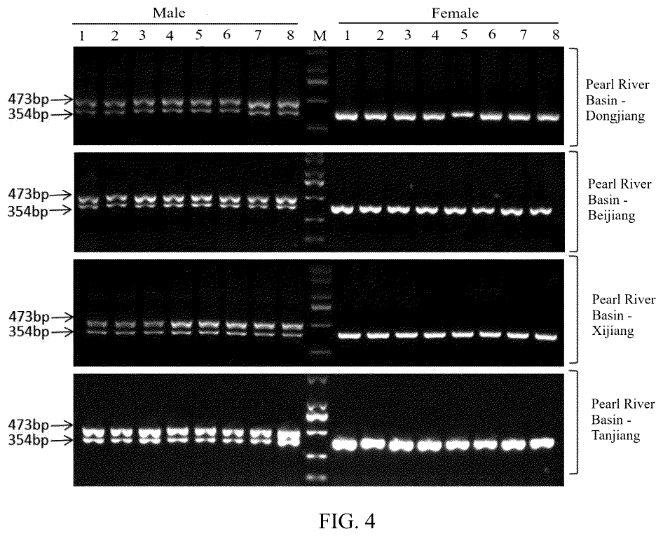

After PCR amplification, the products were subjected to 1.2% agarose gel electrophoresis. Finally, both primer pairs DCQ-1F/R and DCQ-2F/R were able to amplify specific bands in the DNA samples of both male and female individuals. The length of the specific band on the X chromosome produced by DCQ-1F/R was 354 bp, and the length of the specific band on the Y chromosome was 473 bp (see FIG. 4); the length of the specific band on the X chromosome produced by DCQ-2F/R was 649 bp, and the length of the specific band on the Y chromosome was 491 bp. This indicates that the genetic sex molecular markers DCQ-1F/R and DCQ-2F/R can be used to identify the sex of Mastacembelus armatus.

### Embodiment 3

In this embodiment, the genetic sex markers and identification methods provided in 1 and 2 are used for the breeding of all-male Mastacembelus armatus. The process includes the following steps.

(1) The Mastacembelus armatus fry are raised to 20 days old, and fed with eel feed containing 100 mg/kg of the estrogen 17β-estradiol for two months, during which natural light is used, and the water temperature is maintained at 27-29° C., and the fish are fed to satiety.

(2) Using the method from embodiment 2, the Mastacembelus armatus fed with hormones are screened to obtain genetically female fish of the XY type, obtaining pseudo-female fish. Refer to example 2 for specific operations.

(3) The identified XY pseudo-female fish are raised to sexual maturity and mated with normal XY male fish during the breeding season to obtain offspring containing XY and YY male fish.

(4) The YY super-male fish in the offspring are further screened out using male-specific molecular markers through PCR electrophoresis or test-crossing. The obtained YY super-male fish are raised to sexual maturity and mated with XX female fish to obtain all-male fish offspring.

The specific DNA fragments, genetic sex markers, identification methods, and applications provided in the above embodiments of the present application focus on addressing the deficiencies of existing technologies. The WGS sequencing technology and data are used to design four DNA fragments that can complement each other, with good stability and wide applicability, combining in pairs to form two sets of molecular marker primers. This enables the molecular-level identification of the sex of Mastacembelus armatus and is widely applicable for identifying the genetic sex of Mastacembelus armatus in both wild and cultured populations in the Pearl River Basin. This primer pair can rapidly and accurately identify the sex of both male and female Mastacembelus armatus at the molecular level, meeting the requirements for large-scale breeding and production of Mastacembelus armatus in different regions and populations, with low cost and ease of implementation.

The above embodiments are only used to illustrate the present application, and the scope of protection of the present application is not limited to the above embodiments. Those skilled in the art under the embodiments of the present application are within the scope of protection of the present application. Based on the content disclosed in the present application, those skilled in the art of the relevant technical field can achieve the purpose of the present application. Any improvements and modifications made based on the conception of the present application fall within the scope of protection of the present application. The specific scope of protection is determined by the content of the claims.

What is claimed is:

1. A genetic sex marker primer for the identification of the sex of Mastacembelus armatus, wherein the genetic sex marker primer is a primer pair 1, which comprises an upstream primer of Contig-1 and a downstream primer of Contig-1;
nucleotide sequences of the primer pair 1 from 5′-3′ are as follows:
upstream primer of Contig-1:
5′-CCATTCAGCATTACACCATACA-3′;
downstream primer of Contig-1:
5′-TTCCCCTACCAAATCACCA-3′.

2. A genetic sex marker primer for the identification of the sex of Mastacembelus armatus, wherein the genetic sex marker primer is a primer pair 2, which includes an upstream primer of Contig-2 and a downstream primer of Contig-2;
nucleotide sequences of the primer pair 2 from 5′-3′ are as follows:
upstream primer of Contig-2:
5′-TGCTACTGTCCAAAGTAAACAGG-3′;
downstream primer of Contig-2:
5′-CGGGCAGAATCACTCAAA-3′.

3. A method for identifying the sex of Mastacembelus armatus using the genetic sex marker primer according to claim 1, includes the following steps:
S1: designing the primer pair 1;
S2: preparing Mastacembelus armatus DNA sample;
S3: using the DNA sample obtained in S2 as a template, performing PCR amplification with the primer pair 1 designed in S1, and detecting by gel electrophoresis; if the electrophoresis result shows a specific DNA band, the detected Mastacembelus armatus is female; if the electrophoresis result shows two specific DNA bands, the detected Mastacembelus armatus is male.

4. The method for identifying the sex of Mastacembelus armatus using the genetic sex marker primer according to claim 3, wherein the DNA sample preparation in S2 is an extraction of Mastacembelus armatus DNA using a DNA extraction kit.

5. The method for identifying the sex of Mastacembelus armatus using the genetic sex marker primer according to claim 3, wherein in S3, when using the primer pair 1 or the primer pair 2 for PCR amplification, the 25 μL PCR reaction system includes: 100 ng of Mastacembelus armatus DNA sample, 1 μL each of upstream and downstream primers of Contig-1 or Contig-2, 12.5 μL of 2×TaqMasterMix, and ddH2O to make up the system to 25 μL.

6. The method for identifying the sex of Mastacembelus armatus using the genetic sex marker primer according to claim 3, wherein in S3, when using primer pairs for PCR amplification, the PCR reaction program is: denaturation at 95° C. for 3 min; then denaturation at 95° ° C. for 15 s, annealing at 58° C. for 15 s, extension at 72° C. for 1 min, for a total of 20 cycles; then denaturation at 95° C. for 15 s, annealing at 50° ° C. for 15 s, extension at 72° C. for 1 min, for a total of 15 cycles; then final extension at 72° C. for 5 min; in S3, during electrophoresis detection, a 1.2% agarose gel is used to detect the number and length of amplified fragments.

7. The method for identifying the sex of Mastacembelus armatus using the genetic sex marker primer according to claim 3, wherein in S3, the specific band amplified in the electrophoresis detection results indicates female if there is one band, and male if there are two bands; the specific band length of a X chromosome product of the primer pair 1 is 354 bp, and a Y chromosome product is 473 bp.

8. An application of the genetic sex marker primer for Mastacembelus armatus according to claim 1 in all-male breeding of Mastacembelus armatus.

9. A method for identifying the sex of Mastacembelus armatus using the genetic sex marker primer according to claim 2, includes the following steps:
S1: designing the primer pair 2;
S2: preparing Mastacembelus armatus DNA sample;
S3: using the DNA sample obtained in S2 as a template, performing PCR amplification with the primer pair 2 designed in S1, and detecting by gel electrophoresis; if the electrophoresis result shows a specific DNA band, the detected Mastacembelus armatus is female; if the electrophoresis result shows two specific DNA bands, the detected Mastacembelus armatus is male.

10. The method for identifying the sex of Mastacembelus armatus using the genetic sex marker primer according to claim 9, wherein the DNA sample preparation in S2 is an extraction of Mastacembelus armatus DNA using a DNA extraction kit.

11. The method for identifying the sex of Mastacembelus armatus using the genetic sex marker primer according to claim 9, wherein in S3, when using the primer pair 2 for PCR amplification, the 25 μL PCR reaction system includes: 100 ng of Mastacembelus armatus DNA sample, 1 μL each of upstream and downstream primers of Contig-1 or Contig-2, 12.5 μL of 2×TaqMasterMix, and ddH2O to make up the system to 25 μL.

12. The method for identifying the sex of Mastacembelus armatus using the genetic sex marker primer according to claim 9, wherein in S3, when using primer pairs for PCR amplification, the PCR reaction program is: denaturation at 95° C. for 3 min; then denaturation at 95° C. for 15 s, annealing at 58° C. for 15 s, extension at 72° ° C. for 1 min, for a total of 20 cycles; then denaturation at 95° C. for 15 s, annealing at 50° ° C. for 15 s, extension at 72° C. for 1 min, for a total of 15 cycles; then final extension at 72° ° C. for 5 min; in S3, during electrophoresis detection, a 1.2% agarose gel is used to detect the number and length of amplified fragments.

13. The method for identifying the sex of Mastacembelus armatus using the genetic sex marker primer according to claim 9, wherein in S3, the specific band amplified in the electrophoresis detection results indicates female if there is one band, and male if there are two bands; the specific band length of the X chromosome product of the primer pair 2 is 649 bp, and the Y chromosome product is 491 bp.

14. An application of the genetic sex marker primer for Mastacembelus armatus according to claim 2 in all-male breeding of Mastacembelus armatus.


SEQUENCE LISTING (8 sequences)

SEQ ID NO: 1 - DNA, 355 bp, synthetic construct
  source 1..355: mol_type = other DNA
cgctctgggcatggatgaaggagcaagaaagaaactgctcggaggatggggggtgaggcagaaagaactgacacttgacatctgcccctggcccaaaagctacagttacttaatgaggtaactgaagaaagcacccaccaggtgtactctgtaactcattgtcacaaactgggccgcacagcgatgacaaatgagggagatgcacacaaatagctaaactgaaccaattttttatttttaatggaaaagaatattaacccaaagtaatactgaggattaaagatggggtttgcagtcagtaaaatcagctgtgtaggtgatgcaagtgcatgtgtatggtgtaatgctgaatgga

SEQ ID NO: 2 - DNA, 473 bp, synthetic construct
  source 1..473: mol_type = other DNA
gctctggcatggatgaaggagcaagaaagaagctgctcggaggatggggggtgaggcagaaagaacctccagctggatttggagtaaaagagaggaggaaagctaaagccaggagcagatggtcagtgatttggccactacagcagacccaccaactggagagggtcgaaacagttgatggttgggcaccacttgacatctgcccctggcccaaaagctacagttacttaatgaggtaactgaagaaagcacccaccaggtgtactctgtaactcattgtcacaaactgggccgcacagcgatgacaaatgagggagatgcacacaaatagctaaactgaaccaattttttatttttagtggaaaagaatattaacccaaagtaatactgaggattaaagatggtgtttgcagtcagtaaaatcagctgtgtaggtgatgcaagtgcatgtgtatggtgtaatgctgaatgga

SEQ ID NO: 3 - DNA, 22 bp, synthetic construct
  source 1..22: mol_type = other DNA
ccattcagcattacaccataca

SEQ ID NO: 4 - DNA, 19 bp, synthetic construct
  source 1..19: mol_type = other DNA
ttcccctaccaaatcacca

SEQ ID NO: 5 - DNA, 649 bp, synthetic construct
  source 1..649: mol_type = other DNA
agctacgagctagcttaacacctgcgacacctagatcagagaggtctcaagctgcctcacgctgactccagcaacaagcccagcacctgctttaatacacattgctaagtaaaatgtaaaaactcaaatatctatcatattgttaaacggtcaaactatgtctgtttttaggttgagaaacgttagtgttatagttattaaatccgcaagagcgaattacggtggccgacaagggcaaacgcgccgcaacagccaaacacacacaagagagaaacgtgttttggctgttgcggcgcgtttgcccttgtcggccaccgtagataaaatctctataaggaacgaattcgtcaggcttttaggtccacatttgctgaatttgagacggctgttggagtgagttgtcatggtatcaatagacaatctttggctggagagcgcagatcctaacatggcagacatggcggctaaacgtgaactggatagtgttcaacctgatgaattagttcacaaagtgaagaggacggaagctgacaatgatggtggtccaacagttgtaaaggaatcaggctcaggaaatattttgtctgatttccaaatatctggcattttgagtgattctgcccgggagaaaagcatctttatccatgga

SEQ ID NO: 6 - DNA, 491 bp, synthetic construct
  source 1..491: mol_type = other DNA
agctacgagctagcttaacacctgcgacacctagatcagagaggtctcaagctgcctcacgctgactccagcaacaagcccagctcctgctttaataaacattgctaagtaaaatgtaaaaactcaaatatctatcatattgttaaacggtcaaactatgtctgttaggttgagaaacgttagtgttatagttattaaatccgcatttgctgaatttgagacggctgttggagtgagttgtcatggtatcaatagacaatctttggctggagagcgcagaccctaacatggcagacatggcggctaaacgtgaactggatagtgttcaacctgatgaattagttcacaaagtgaagaggacggaagctgacaatgatggtggtccaacagttgtaaaggaatcaggctcaggaaatattttgtctgatttccaaatatctgccattttgagtgattctgcccgggagaaaagcatctttatccatggaaag

SEQ ID NO: 7 - DNA, 23 bp, synthetic construct
  source 1..23: mol_type = other DNA
tgctactgtccaaagtaaacagg

SEQ ID NO: 8 - DNA, 18 bp, synthetic construct
  source 1..18: mol_type = other DNA
cgggcagaatcactcaaa